(19) United States
(12) Patent Application Publication
(10) Pub. No.: US 2020/0289428 A1
(43) Pub. Date: Sep. 17, 2020

(54) METHODS OF PROTECTING THE EYE HEALTH OF CHILDREN EXPOSED TO BLUE AND WHITE LIGHT

(71) Applicant: KEMIN INDUSTRIES, INC., DES MOINES, IA (US)

(72) Inventors: Joanne A. Lasrado, Des Moines, IA (US); Brandon J. Lewis, Johnston, IA (US); Samanta Maci, Belas (PT); Richard Roberts, Johnson, IA (US); Kelli Herrlinger, Ankeny, IA (US)

(21) Appl. No.: 16/819,061

(22) Filed: Mar. 14, 2020

Related U.S. Application Data
(60) Provisional application No. 62/819,279, filed on Mar. 15, 2019.

(51) Int. Cl. A61K 31/047; A61P 27/02

(52) U.S. Cl. CPC A61K 31/047; A61P 27/02

(57) ABSTRACT

The present invention relates generally to a method of reducing the impact on visual performance resulting from exposure to harmful light rays in children through supplementation with lutein and/or zeaxanthin, in particular protecting the eye from damage created due to exposure to blue light and/or white light.

### CROSS-REFERENCE TO RELATED APPLICATION

This application claims the benefit of priority to U.S. Provisional Patent Application No. 62/819,279, filed Mar. 15, 2019, entitled “METHODS OF PROTECTING THE EYE HEALTH OF CHILDREN EXPOSED TO BLUE AND WHITE LIGHT,” the disclosure of which is hereby incorporated herein by reference in its entirety.

### BACKGROUND OF THE INVENTION

Blue light is the prevalent emission radiation among solar light and LED technology from digital devices such as smartphones, computers and many TVs screens, and is one of the most damaging wavelengths for the retina. Little is known regarding the long-term effects of exposure to blue light, particularly for school-aged children that may be exposed to blue light during the school day on computer screens, as well as additional screen time that may occur in the evenings and over the weekends.

Visible light, or white light, comprises the region of light within the wavelengths of 400-700 nm in the electromagnetic spectrum. Blue light is the portion of the electromagnetic spectrum in the visible region with wavelengths ranging from 400-500 nm. The wavelengths of blue light are close to longer ultraviolet wavelengths, typically called the UVA spectrum (315-400 nm), and the blue region of the visible spectrum is particularly important because it still has high energy (even though lower than UV) and longer wavelengths that can penetrate tissue deeper than UV light. Godley B. F., Shamsi F. A., Liang F. Q., Jarrett S. G., Davies S. and Boulton M., Blue light induces mitochondrial DNA damage and free radical production in epithelial cells, J. Biol. Chem. 280 (2005) 21061-21066; Opländer C., Hidding S., Frauke B., Werners F. B., Born M., Pallua N. and Suschek C. V. Effects of blue light irradiation on human dermal fibroblasts. Journal of Photochemistry and photobiology B: Biology. 2011. 103: 118-125.

The adverse effects of blue light on a variety of cell lines were studied in the past by various research groups and a few of them were briefly summarized by Oplander et al. Particular focus was given to the penetration of blue light into retinal cells and its effect on the generation of age related macular degeneration was studied by many research groups. There remains a need, however, to better understand the short and long-term effects of children exposed to blue and white light. One cross-sectional study conducted in children up to 10 years of age suggests that prolonged use of videogames may have an impact on reporting of asthenopia and refractive errors, however to our knowledge no intervention studies have evaluated the impact of exposure to blue light and white light on visual function and in this demographic. Rechichi C, De Mojà G, Aragona P., Video Game Vision Syndrome: A New Clinical Picture in Children? J. Pediatr Ophthalmol Strabismus. 2017 Nov. 1; 54(6):346-355.

### SUMMARY OF THE PRESENT INVENTION

The present invention relates generally to a method of reducing the impact on visual performance resulting from the exposure to harmful light rays, in particular blue and white light and at the same time protecting the eye. Visible light is the part of the electromagnetic spectrum in the range from 400 to 780 nm that is perceived by the human eye. This light is seen as “white” when it is composed by all the wavelengths of the visible spectrum. Blue light contains wavelengths of the visible spectrum at 400-500 nm which are close to the UVA spectrum. The blue region (400-500 nm) is also known as high-energy visible (HEV) light because of the high energy relative to other wavelengths of the visible spectrum although lower than UV. This is important because although less energetic that UV, blue wavelengths are longer and can penetrate tissue deeper than UV light.

The sun is the primary source of white light and blue light. The sun's spectrum peaks over the visible light range and almost 30% of the visible spectrum is represented by blue light. In more recent years the human eye has also been increasingly exposed by blue light from white-light LED lighting devices (blue LED and yellow phosphor) such as smartphones, tablets, laptops and video games. Scientific Committee on Health, Environmental and Emerging Risks (SCHEER) Preliminary opinion on potential risks to human health of light emitting diodes (LED), European Union 6 Jul. 2017.

Plant-derived carotenoids lutein and zeaxanthin are incorporated into ocular tissues where they function to absorb visible light and thus protect the retina. The inventors surprisingly found that supplementation of lutein and zeaxanthin in children, such as those children under the age of 18, for instance toddlers (2 or 3 years of age) through adolescents or teenagers, and in at least one embodiment children between 7 to 12 years of age, could provide some protection on reducing the impact on visual performance resulting from exposure to either blue or white light and thus attenuating the potential damage resulting from this exposure.

### BRIEF DESCRIPTION OF THE DRAWINGS

FIG. 1 depicts the Study Flow Diagram (Example 1).

FIG. 2 depicts the sum of all questions for the Visual Analog Scales (VAS) for Eye Strain and Fatigue for placebo and lutein groups, immediately following blue light exposure.

FIG. 3 depicts the sum of all questions for the Visual Analog Scales (VAS) for Eye Strain and Fatigue for placebo and lutein groups, immediately following white light exposure.

FIG. 4 depicts the reported raw score from the Visual Analog Scales (VAS) for Eye Strain and Fatigue for the question “Are you sensitive to the light” for placebo and lutein groups, immediately following blue light exposure

FIG. 5 depicts the change from baseline in the average of 3 glare recovery (seconds) measurements over 112-day supplementation with Lutein/Zeaxanthin supplement or placebo (n=12). Values are means±SEMs.

### DETAILED DESCRIPTION OF EMBODIMENTS

The present invention relates to administering compositions containing lutein and/or zeaxanthin in order to reduce or minimize the impact on visual performance resulting from exposure to light and consequently also providing protection to the eye, for instance through oral administration.

The human eye is a highly complex organ influencing all aspects of life. Natural sunlight is essential for vision and provides many psychological and physiological benefits. Yet there is caution because ultraviolet (UV) radiation from sunlight can damage the eye. Berman K, Brodaty H. Psychosocial effects of age-related macular degeneration. Int Psychogeriatr. 2006; 18(3):415-428. Much of the UV radiation that reaches the eye is filtered by the cornea, as a natural form of protection. UVA, is the lowest energy band of UV radiation and is the closest to the visible light range. This range is known to penetrate the cornea and reach the lens and retina of the eye. While in adults all the UVA are filtered by the cornea or the lens, in children approximately up to the age of 9 the lens permits transmission of 2-5% of UV at 320 nm to the retina. Scientific committee on emerging and newly identifies health risk (SCENHIR). Health effect of artificial Light. European Union, 2012. http://ec.europa.eu/health/scientific_committees/policy/index_en.htm. The highest-energy visible light, close to the damaging UVA band is the blue-violet range. It is thought that over-exposure to these ranges of wavelength (UVA and blue-violet light) on the eye could be a contributing factor to macular degeneration. Similar to what has been observed for UV, vulnerability of children to blue light is particularly high considering that approximately 15% of light at 400 nm and 65% at 460-480 reaches the retina, compared to only 1% and 40% in older adults, respectively.

Blue wavelengths of light have been shown to be especially detrimental to visual performance. Visual performance is compromised when glaring light enters the eye. Central vision can be most affected since intense light imaged onto the fovea causes discomfort. The fovea is responsible for the highest visual performance for almost all vision parameters so that any factor that negatively impacts the fovea will result in reduced visual performance. Intense lights in the periphery can also be scattered by the ocular media over the fovea and result in reduced contrast for objects viewed centrally therefore reducing the ability to see an object within a background.

Yellow filters have been demonstrated to improve aspects of vision including contrast particularly under blue light conditions. As such, intraocular yellow filters can be congenitally found in many species of animals. For instance, in primates, a yellow spot exists in the center of the fovea known as the macula lutea (macula). The macula is only 4% of the central area of the retina, yet responsible for photopic vision, due to its high density of cone photoreceptor cells. Coleman H, Chan C, Ferris F 3rd, Chew E. Age-related macular degeneration. Lancet. 2008; 372 (9652):1835-1845. The macular area of the eye contains the oxygenated carotenoids lutein and zeaxanthin, known as the macular pigment, that behave as blue light filters and antioxidants to help promote visual function and protect the photoreceptor cells against damaging blue light. Studies supplementing adults with lutein and zeaxanthin and exposing them to a source of bright light have shown a significant increase in the macular pigment optical density and improvements in glare, contrast sensitivity, and photo stress recovery time.

Blue light exposure has received more attention with the advancement in technology due to the high demand for efficient energy light sources and computational processing in everyday life. Tosini G, Ferguson I, Tsubota K. Effects of blue light on the circadian system and eye physiology. Mol Vis. 2016; 22:61-72. Blue light sources are present in high brightness light emitting-diodes (LEDs), white or blue in color, found in many new, energy efficient LED light bulbs. This source is also generated from solid-state lighting (SSL) and can be found in smart phones, laptop computers, iPads, tablets, e-readers and large flat screen television sets. Although there is controversy in the literature regarding whether the amount of blue light emitted from these devices is sufficient to cause short-term photoreceptor damage in humans, current society utilizes these devises extensively and the potential exists for long-term damage with chronic usage. Experimental in-vitro studies suggest that cumulative blue and white light exposure below the levels causing an acute effect can also induce photochemical retinal damage. Furthermore, animals exposed to intense light with blue-bbcking filter show significantly more conserved visual responses, retinal structure and photoreceptor survival than animals exposed to unfiltered light. Tejedor et alPLoS One. 2018 Mar. 15; 13(3). It is established, however, that LEDs do cause issues in terms of flicker, dazzle, distraction discomfort and glare. Children have a higher sensitivity to blue light radiation, so blue light can be very dazzling for young children. Some LED emission spectra can induce photochemical retinopathy, which is a concern especially for children below three years of age.

Sunlight is the most significant source of blue light; however, it is expected that exposure to these wavelengths of the visible spectrum will increase in the US by roughly 800% over the next four years, in part due to the increased exposure to digital devices. American Optometric Association. Eye-Q Survey (Internet). American Optometric Association; 2015, available at: http://www.aoa.org/documents/newsroom/2015_AmericanEye-Q_surveyresults.pdf. Greater than half (65%) of children spend two or more hours on digital devices daily. Furthermore, 70% of parents who let their children spend three or more hours on digital devices each day reported that they are very or somewhat concerned about the impact of digital devices on eye health. The Vision Council. Eyes Overexposed: The Digital Device Dilemma [Internet]. The Vision Council; available at http://www.thevisioncouncil.org/sites/default/files/2416_VC_2016 EyeStrain_Report_WEB.pdf. This exposure can be higher in some children, where 15% of parents who have children do not limit their device time, see Eustis S. LED Lighting-Markets at $4.8 Billion in 2012 Reach $42 Billion by 2019 [Internet]. Lexington, Mass.: Wintergreen Research Inc.; 2013, available at: http://wintergreenresearch.com/reports/LED %202013%20press %20release.pdf, and 24% and 26% of children get their first smartphone or tablet at 6-8 years old or 9-11 years old, respectively. American Optometric Association. Eye-Q Survey (Internet). American Optometric Association; Available: http://www.aoa.org/documents/newsroom/2015_AmericanEye-Q._surveyresults.pdf. These significant changes in blue light exposure have evolved within the last decade and are increasing exponentially; however, there is still little to no information available about methods to protect against damage caused by exposure to blue light in children, particularly the potential benefits of lutein supplementation in order to protect the visual response to blue light exposure in children.

### Example 1

The inventors evaluated the effects of lutein supplementation on visual performance following blue light and white light exposure in children. This was a single-center, randomized, double-blind, placebo-controlled, parallel, pilot study. The study consisted of a single 112-day study period.

The investigators considered the following factors: (1) The difference in the change from baseline to 112 days of supplementation, between placebo and lutein, in Visual Analog Scale (VAS) for Eye Strain and Fatigue scores, immediately following either blue light or white light exposure; (2) The differences in VAS for Eye Strain and Fatigue scores for change from baseline to 112 days of supplementation following blue light exposure and the change from baseline to 112 days of supplementation following white light exposures in placebo and lutein groups; and (3) The difference in the change from baseline to 112 days of supplementation, between placebo and lutein treatment in the time of glare recovery.

Materials and Methods

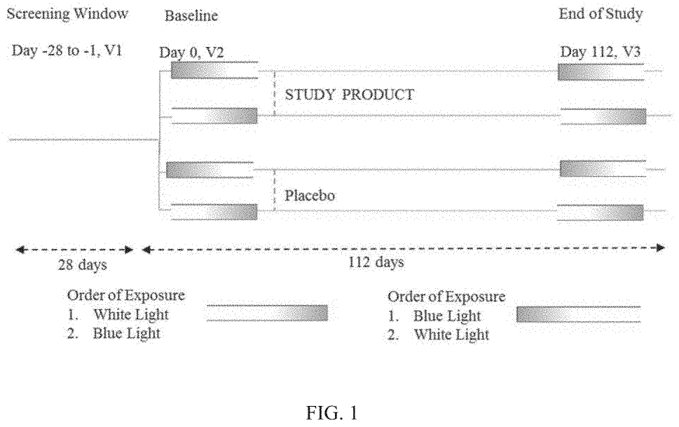

This was a single-center, randomized, double-blind, placebo-controlled, parallel study. Twelve healthy school-aged children were planned to be randomized, with 6 participants randomized equally to each of the two study-arms in a double-blinded manner at a ratio of 1:1. Within each group, the participants were equally randomized into two different orders of exposure, as described in FIG. 1, White Light→Blue Light and Blue Light→White Light, respectively.

The randomized controlled trial is a rigorous design that allows the investigation of the impact of an investigational product on study outcomes by controlling bias and limiting confounding factors. Participants and the study investigator(s) were blinded to minimize any influence on study measurement and interpretation.

The control group consisted of volunteers drawn from the same recruitment pool who met all inclusion and no exclusion criteria. The placebo product was matched to the investigational product and contained similar excipient to ensure blinding.

At screening, participants were deemed healthy by medical history and physical exam, vital signs, hematology and clinical chemistry. A food frequency questionnaire was administered to determine eligibility based on carotenoid intake and a visual assessment was performed by an optometrist. Eligible participants returned for their baseline visit and were randomized into either the Lutein/Zeaxanthin supplementation arm or the placebo arm. Visual performance evaluations were conducted. Participant's self-reported eye strain and fatigue via a visual analog scale (VAS) questionnaire were obtained after being placed in blue and white light. Photostress recovery time was assessed following a 10-second exposure to a dual glare source. Visual acuity was re-established between assessments. A compliance phone call was conducted to ensure compliance and that subjects completed their study diaries. On Day 112 participants were assessed for, VAS Eye Strain and Fatigue and glare recovery times.

The planned sample size for this study was 12 participants, with 6 participants randomized to each Lutein/Zeaxanthin and placebo groups. Thirteen healthy school-aged children were recruited and completed the study. All individuals were included in the safety analysis. However, only 12 were included in the per protocol statistical analysis due to poor compliance by one individual. The apriori per protocol (PP) population definition was assessment was conducted comprised of participants who consumed at least 80% of the either study product dose, did not have any major protocol violations (those that were deemed to have possible effects on the outcomes) and completed all study visits. Due to the small sample size and “pilot trial” status of this study the inventors considered a p<0.1 as significant for consideration.

For consideration to be included in the study, children needed to be 8-12 years of age, healthy, and have 20/20 vision in both eyes as assessed by optometrist and a body mass index-for-age-percentile at 3% and below 97%. In addition, children should have a and low carotenoid (defined as ≤2 mg/day) intake as assessed by their Food Frequency Questionnaire.

The participants were divided into two groups; the first group was administered the investigational product, or study product, while the second group received the placebo. The product and placebo were orally administered. The investigational product, Lutein Softgel, consists of the ingredients seen in Table 1.

TABLE 1
Active ingredients and dosages in Lutein Softgel

| | Quantity | Active Ingredient |
|---|---|---|
| Dietary Ingredient | (per Softgel) | (per Softgel) |
| Lutein in Safflower Oil | 57.5 mg | 10 mg lutein, 0.8 mg |
| | | zeaxanthin |
| Zeaxanthin in Safflower Oil | 9.86 mg | 1.2 mg zeaxanthin |

Non-active ingredients: caramel, gelatin, glycerin, safflower oil, and water

The placebo contained caramel, gelatin, glycerin, safflower oil, and water. No differences in size, color, taste, texture, or packaging were detectable between the two products. All study products were made identical in appearance (size, color, taste, texture, and packaging) to make them indistinguishable to participants and study personnel.

Demographics at Screening:

Participants ranged from 7 to 12 years of age and 58% were female. Participants were predominantly non-Hispanic whites (67%). All participants were deemed healthy. There were no other significant between-group differences observed in safety outcomes at screening.

Compliance:

The mean compliance was 98.82% for Lutein/Zeaxanthin and 90.74% for placebo and was not significantly different between groups.

Results:

Eye Strain and Fatigue Assessed in Blue and White Light

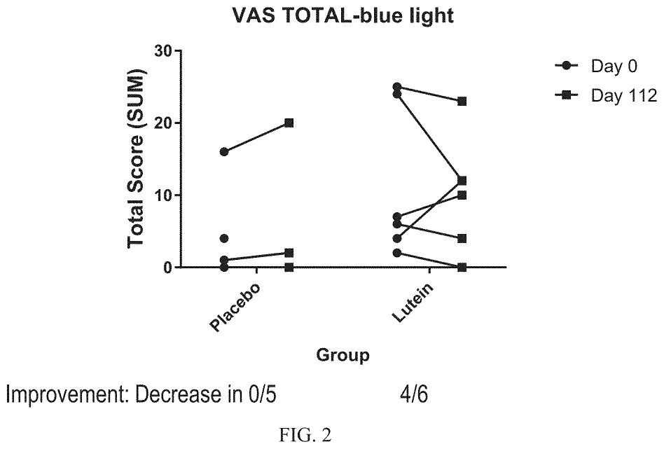

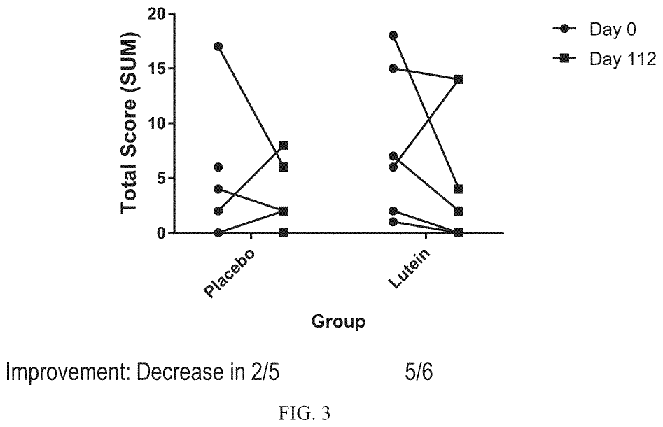

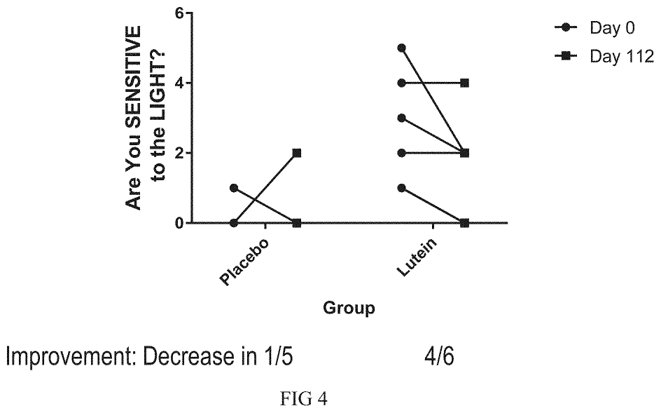

The total sum for VAS Eye Strain and Fatigue overall score when children were exposed to blue light decreased (where a decrease is indicative of improvement) in 4 out of 6 children taking the study product in comparison to the placebo group where no subjects observed an improvement (FIG. 2). Furthermore, the total sum for VAS Eye Strain and Fatigue overall score (where a decrease is indicative of improvement) was observed when children taking the study product were exposed to white light. This decrease/improvement was found for 5 out of 6 children taking the study product group in comparison to the placebo group where only 2 individuals observed an improvement (FIG. 3). Evaluation of the questions within the VAS Eye Strain and Fatigue found that when subjects were asked “Are you sensitive to light”, following blue light exposure 4 out of 6 individuals in the study product group reported less sensitivity to light in comparison to only 1 individual in the placebo group (FIG. 4).

Secondary Outcome: Glare Recovery

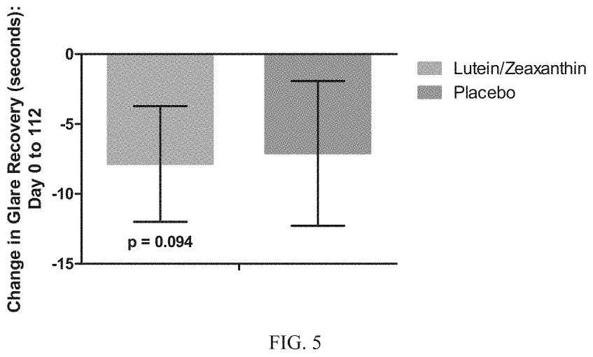

Within the Lutein/Zeaxanthin group, there was a significant improvement in glare recovery time by 64% at Day 112 (p=0.094, FIG. 5), but the magnitude of change from baseline was not significantly different between groups. Furthermore, all 6 subjects in the Lutein group had improvements in glare recovery.

### DISCUSSION

Children are more sensitive to the effects of blue light than adults because of the transparency of the clear crystalline lenses of their eyes, which allows a broader waveband of light to reach the retina than in adult eyes. Behar-Cohen F, Martinsons C, Vienot F, Barlier-Salsi A, Cearini J, Enouf O, Garcia M, Picaud S, and Attia D (2011). Light-emitting diodes (LED) for domestic lighting: Any risk for the eye? Prog Retin Eye Res. 30: 239-257. This broader waveband of light includes the longer wavelength ultraviolet light (namely some longer UVA wavelengths) and the shorter wavelength visible light (violet and blue wavelengths) that are typically reduced by the yellowness of the lenses of the eye that are found in adults. Behar-Cohen, et al. (2011) ibid. and Dillon J, Zheng L, Merriam J, and Gaillard E (2004). Transmission of light to the aging human retinal: possible implications for age related macular degeneration. Exp Eye Res. 79: 753-759. Furthermore, the shorter axial length of a child's eye could, at least potentially, result in less dissipation of light energy/intensity before these potentially harmful wavelengths of light reach the retina as compared to the adult eye. However, only a very limited number of studies have been conducted to demonstrate the effects of light in the childhood eye. That lack of information and absence of studies leaves significant gaps in the understanding of the effects of light on the childhood eye. Understanding the effects of that light, especially the effects resulting from exposure to the blue wavelengths of light in children is more important than ever, given that children tend to have longer screen time exposures than are recommended for healthy pediatric ocular development. Anderson S, Economos C, Must A (2008). Active play and screen time in US children aged 4 to 11 years in relation to sociodemographic and weight status characteristics: a nationally representative cross-sectional analysis. BMC Publ Health. 8: 366.

The macular pigment helps protect the retina from light-induced damage through its antioxidant and anti-inflammatory potential while also absorbing some of the wavelengths of light reaching the posterior tissue that could cause damage to the retina, especially light wavelengths in the blue region of the visible spectrum. Kijlstra A, Tian Y, Kelly E, Berendschot T (2012). Lutein: more than just a filter of blue light. Prog Retin Eye Res. 31: 303-315. Tosini G, Ferguson I, and Tsubota K (2016). Effects of blue light on the circadian system and eye physiology. Mol Vis 22: 61-72. Studies conducted in-vitro and in-vivo have demonstrated that supplementation with lutein and zeaxanthin help protect the retinal tissues against the negative effects of light, especially the blue wavelengths of light. Bian Q Gao S, Zhou J, Qin j, Taylor A, Johnson E, Tang G, Sparrow J, Gierhart D, and Shang F (2012). Lutein and zeaxanthin supplementation reduces photooxidative damage and modulates the expression of inflammation-related genes in retinal pigment epithelial cells. Free Radic Biol Med. 53: 1298-1307. Barker F, Snodderly D, Johnson E, Schalch W, Koepcke W, Gerss J, and Neuringer M (2011).

Nutritional manipulation of primate retinas, V: effects of lutein, zeaxanthin, and n-3 fatty acids on retinal sensitivity to blue-light-induced damage. Invest Ophthalmol Vis Sci. 52: 3934-3942.

Some additional benefits of supplementation with lutein and zeaxanthin have been demonstrated in adult subjects, including improvements in visual parameters of subjects that spend a considerable amount of time exposed to sources of blue light such as screens of digital devices. Stringham J, Stringham N, and O'Brien K (2017). Macular Carotenoid Supplementation Improves Visual Performance, Sleep Quality, and Adverse Physical Symptoms in Those with High Screen Time Exposure. Foods. 6: pii: E47. These benefits include an improvement in contrast sensitivity, glare, and photostress recovery. Furthermore, a reduction in headache frequency, eye strain and eye fatigue were also found in subjects supplemented with lutein and zeaxanthin. The improvements observed in contrast sensitivity, glare and photostress recover in this adult population were expected since prior studies conducted in young adults without any evidence of ocular disease had been previously demonstrated. Stringham J and Hammond B (2008). Macular pigment and visual performance under glare conditions. Optom Vis Sci. 85: 82-88. Hammond B, Fletcher L, Roos F, Wittwer J, and Schalch W (2014). A double-blind, placebo-controlled study on the effects of lutein and zeaxanthin on photostress recovery, glare disability, and chromatic contrast. Invest Ophthalmol Vis Sci. 55: 8583-8589. However, no studies had previously been conducted on the effects of lutein and zeaxanthin supplementation on headache frequency, eye strain, or eye fatigue that could be definitely related to an effect induced specifically by these carotenoids.

Although the current knowledge of the effects that the macular pigment (lutein and zeaxanthin in the eye) have revealed a myriad of benefits when evaluated in the eyes of young to elderly adults, these results cannot be extended to the eyes of children for several reasons. First and foremost, the eyes of children, especially younger children, are not yet fully mature thereby making any type of extrapolation difficult. Secondly and perhaps equally importantly, no studies similar to the ones described above have been conducted in a childhood population. In fact, no vision-related studies have been conducted in individuals younger than 18 years of age. This lack of information motivated the inventors to conduct the randomized, double-blind, placebo-controlled pilot study, which resulted in unexpected and surprising results.

The inventors evaluated the effect lutein supplementation on visual performance and related ocular parameters associated with blue light or white light exposure had upon school-aged children. Thirteen healthy children with healthy eyes as demonstrated by a professional eye examination at the beginning of the study and who were between the ages of 7 to 12 years with a BMI 15.12-22.12 kg/m² were enrolled in and completed the study. Participants were predominantly non-Hispanic, Caucasian of Western European decent. The randomization yielded two test groups that exhibited no differences in demographics, blood pressure, BMI or heart rate at baseline. Participants were required to consume one Lutein/Zeaxanthin Softgel (Lutein/Zeaxanthin) or a placebo every morning with breakfast for the 4-month study period (112 days).

Although the long-term effects of continued digital device screen exposure are not currently known, there is a growing concern that the close proximity of those screens to the eyes combined with the considerable amount of time spent in front of these light emitting screens could negatively impact vision and visual quality. Perhaps more importantly, these effects could be greater in children than adults because of the clarity of the lenses of the eye, which would allow more damaging light to pass through to the retina. The blue wavelengths of light are the prevalent emission among smartphones, computers and many TVs, and exhibit the greatest damaging potential of any wavelengths within the visible spectrum. Sheppard A and Wolffsohn J (2018). Digital eye strain: prevalence, measurement and amelioration. BMJ Open Opthalmology. 3: e000146. Research suggests that prolonged exposure to blue light may cause damage to retinal cells. Such damage over time could result in vision problems such as macular degeneration and cataracts. This is where the plant-derived carotenoids, lutein and zeaxanthin, become important. They are physiologically incorporated into ocular tissues where they function to absorb visible light and thus influence the optical characteristics of the human eye. Hammond B. Carotenoids. Adv Nutr. 2013; 4(4):474-476.

Although there is a significant body of scientific literature on lutein and zeaxanthin supplementation in older adults and a growing number of publications in younger adults, there is a paucity of data on the effects that these carotenoids might have in the eyes of children. The inventors findings add valuable data to the knowledge base as the first study to show improvement in VAS total score (blue light and white light) for eye strain and fatigue, contrast sensitivity (blue light), and glare recovery (blue light) following light exposure in children.

Several undesirable outcomes are associated with excessive screen time, and augmentation of the macular pigment via supplementation has been proposed to improve visual performance and ameliorate adverse outcomes in adults. Visual processing speed, assess as temporal contrast sensitivity function (tCSF), was found to increase in young healthy adults aged 18-32-year-old adults after a 4-month lutein/zeaxanthin supplementation period. Bovier E and Hammond B (2015). A randomized placebo-controlled study on the effects of lutein and zeaxanthin on visual processing speed in young healthy subjects. Arch Biochem Biophys. 572: 54-57. Furthermore, daily carotenoid supplementation for six months resulted in significant improvement in macular pigment optical density and visual performance measures, versus placebo. Stringham J, Stringham N, and O'Brien K (2017). Macular Carotenoid Supplementation Improves Visual Performance, Sleep Quality, and Adverse Physical Symptoms in Those with High Screen Time Exposure. Foods. 6: pii: E47. The underlying mechanism for that improvement was identified as an augmentation of macular pigment by lutein and zeaxanthin resulting in increasing visual performance. The selection of young healthy adults in both studies is especially significant as vision in this age group has been traditionally considered to be at peak efficiency and therefore thought to demonstrate resilience to change due to ceiling effects. Bovier E and Hammond B (2015). A randomized placebo-controlled study on the effects of lutein and zeaxanthin on visual processing speed in young healthy subjects. Arch Biochem Biophys. 572: 54-57. The relationships between the amount of lutein and zeaxanthin in the eye with visual parameters (tCSF and visual performance) are a result of the ability of the macular pigment (the lutein and zeaxanthin in the eye) and the amount of blue light that is impinged upon the photoreceptors in the retina. Renzi L and Hammond B (2010). The effect of macular pigment on heterochromatic luminance contrast. Exp Eye Res. 2010 December; 91(6):896-900. Renzi L and Hammond B (2010). The relation between the macular carotenoids, lutein and zeaxanthin, and temporal vision. Ophthalmic Physiol Opt. 30: 351-357.

Results of the current Lutein/Zeaxanthin study in children showed that glare recovery time improved significantly from baseline to Day 112 by 64% with Lutein/Zeaxanthin supplementation (FIG. 5). This is a new, unique discovery since glare/photostress recovery has not been previously reported in children. Glare/photostress recovery times can vary greatly based on the age of the participant and the distance from and wavelength composition of the glare source in adults. Stringham J and Hammond B (2007). The glare hypothesis of macular pigment function. Optom Vis Sci. 84: 859-864. Stringham J and Hammond B (2008). Macular pigment and visual performance under glare conditions. Optom Vis Sci. 85: 82-88. Stringham J, Garcia P, Smith P, McLin L, and Foutch B (2011). Macular Pigment and Visual Performance in Glare: Benefits for Photostress Recovery, Disability Glare, and Visual Discomfort. Invest Ophthalmol Vis Sci. 52: 7406-7415. However, glare recovery times in children in this study are consistent with those reported in the literature, where the average photostress recovery time has been reported to be between 8 to 70 seconds. Stringham J and Hammond B (2007). The glare hypothesis of macular pigment function. Optom Vis Sci. 84: 859-864.

The American Academy of Pediatrics recommends no more than 2 hours of screen time per day for children. However, according to nationally representative cross-sectional US study, 5% of children aged 4-11, spend, on average, more than 2 hours per day in front of a computer or television screen. Anderson S, Economos C, and Must A (2008). Active play and screen time in US children aged 4 to 11 years in relation to sociodemographic and weight status characteristics: a nationally representative cross-sectional analysis. BMC Publ Health. 8:366. The proportion of children spending more time in front of light emitting screens is much higher by the time that children reach the age of 10 where it has been found to be over 70% in children of both genders. The relationship between age and screen time is curvilinear, depicted by rising levels in early childhood with a peak at about age 9 to 12 years of age and with decreasing levels in adolescence. Marshall S, Gorely T, Biddle S (2006). A descriptive epidemiology of screen-based media use in youth: a review and critique. J Adolesc. 29: 333-349. This underscores the importance of protection against eye damage since the time spent in front of a screen, socially and/or associated with education, is unlikely to decline in this population as technology advances.

Visual fatigue, eye weakness, or eye strain are subjective sensations classified as asthenopia [Vilela M, Pellanda L, Fassa A, Castagno V (2015). Prevalence of asthenopia in children: a systematic review with meta-analysis. J Pediatr. 91: 320-325.]. While symptoms are usually of a transient nature, the condition can cause frequent and potentially significant discomfort for sufferers. A recent systematic review and meta-analysis of population-based prevalence studies estimated the prevalence of asthenopia in children to be 19.7%. Additionally, it has been reported that children between the ages of 3 and 10 years who spent more than 30 minutes per day in front of video game screens were significantly more likely to experience asthenopia than children who spent less than 30 minutes per day. Rechichi C, De Mojà G, and Aragona P (2017), Video Game Vision Syndrome: A New Clinical Picture in Children? J Pediatr Ophthalmol Strabismus. 54: 346-355. The current study in school-aged children revealed an increase in number of subjects within the lutein/zeaxanthin group reporting an improvement in self-reported eye strain and eye fatigue, as assessed by a questionnaire, after 4 months of lutein/zeaxanthin supplementation following exposure to blue and/or white light. Furthermore, sensitivity to light was reduced following blue light exposure in those individuals who consumed lutein and zzeaxanthin. These data are indicative of less discomfort in children following screen time related light exposure resulting from lutein supplementation.

Digital eye strain is characterized by a range of ocular and visual symptoms and accommodative behaviors. Estimates suggest that the prevalence among computer users may be 50% or more. Sheppard A and Wolffsohn J (2018). Digital eye strain: prevalence, measurement and amelioration. BMJ Open Opthalmology. 3: e000146. Other indicators of excessive screen time that are more often examined in adults but may be interesting in a pediatric population are headaches, blurry vision and neck strain, in addition to improvements in macular pigment optical density and visual performance measures. Stringham et al. (2017) reported significant improvements in indicators of excessive screen time; overall sleep quality, headache frequency, eye strain and eye fatigue in young adults with daily carotenoid supplementation for 6 months. Stringham J, Stringham N, and O'Brien K (2017). Macular Carotenoid Supplementation Improves Visual Performance, Sleep Quality, and Adverse Physical Symptoms in Those with High Screen Time Exposure. Foods. 6: pii: E47. According to a report on digital eye strain published by the Vision Council, individuals report suffering from physical discomfort after screen use for longer than two hours at a time. Barlow S and Expert Committee (2007). Expert committee recommendations regarding the prevention, assessment, and treatment of child and adolescent overweight and obesity: summary report. Pediatrics. 120 Suppl 4:S164-92. Although the long-term effects of more than two consecutive hours of screen time in school aged children are not yet fully understood, this pattern is not uncommon in this age group. Therefore, it is likely that such issues could also exist in children thereby creating a need for amelioration.

Visual performance measures are the logical parameters to examine in young children, as parents and children are hesitant in participating in studies requiring invasive procedures such as blood sampling. Results from the adult population have revealed interesting relationships between biological markers and visual performance measures. Hammond et al. (2014) showed the benefits of lutein/zeaxanthin supplementation in healthy young adults over a 1-year period as measured by an increase in serum levels of these two carotenoids as well as changes in macular pigment optical density. That study found a positive relationship between serum levels of lutein/zeaxanthin and macular pigment optical density benefits and visual performance parameters including contrast sensitivity, photostress recovery and glare tolerance. Hammond B, Fletcher L, Roos F, Wittwer J, and Schalch W (2014). A double-blind, placebo-controlled study on the effects of lutein and zeaxanthin on photostress recovery, glare disability, and chromatic contrast Invest Ophthalmol Vis Sci. 55: 8583-8589.

Nolan et al. (2016), found that significant changes in contrast sensitivity were not observed before 12 months of supplementation, however statistically significant increases in serum lutein, zeaxanthin, and meso-zeaxanthin, and in macular pigment were observed by three months. Nolan J, Power R, Stringham J, Dennison J, Stack J, Kelly D, et al (2016). Enrichment of Macular Pigment Enhances Contrast Sensitivity in Subjects Free of Retinal Disease: Central Retinal Enrichment Supplementation Trials—Report 1. Invest Ophthalmol Vis Sci. 57: 3429-3439. These results are in contrast to those of Hammond, et al. (2014) who found that 12 months of supplementation with lutein/zeaxanthin increased serum carotenoid levels and macular pigment optical density along with glare tolerance, photostress recovery, and chromatic contrast Hammond B, Fletcher L, Roos F, Wittwer J, and Schalch W (2014). A double-blind, placebo-controlled study on the effects of lutein and zeaxanthin on photostress recovery, glare disability, and chromatic contrast. Invest Ophthalmol Vis Sci. 55: 8583-8589. Regardless of the results found in an adult population, these parameters may serve as early indicators of changes that precede improvements in visual performance measures such as contrast sensitivity in any age-related population, including children, and could help to identify those individuals that may benefit most from supplementation. It is important to realize that our knowledge of how visual biomarkers correlate with performance measures has not been adequately explored in the pediatric population.

In addition to placing a child at risk for early eye damage, it is important to note that excessive screen time can impact a young child's physical, social, emotional, and cognitive development Sleep quality and duration has declined over time, with adverse health consequences of sleep deprivation. Chang A, Aeschbach D, Duffy J, and Czeisler C (2015). Evening use of light-emitting eReaders negatively affects sleep, circadian timing, and next-morning alertness. Proc Natl Acad Sci U A. 112: 1232-1237. Literature supports an adverse relationship between screen time and sleep outcomes in children and adolescents. Magee C, Lee J, and Vella S (2014). Bidirectional relationships between sleep duration and screen time in early childhood. JAMA Pediatr. 168: 465-470. Hale Land Guan S (2015). Screen Time and Sleep among School-Aged Children and Adolescents: A Systematic Literature Review. Sleep Med Rev. 21: 50-58. Furthermore, according to a cross-sectional study of the National Health and Nutrition Examination Survey (NHANES), screen time is positively correlated with caffeine intake. Ahluwalia N, Frenk S, and Quan S (2018). Screen time behaviours and caffeine intake in US children: findings from the cross-sectional National Health and Nutrition Examination Survey (NHANES). BMJ Paediatr Open. 2: e000258. This also adds strength to the negative impact of screen time on sleep quality, as well on an aspect of dietary quality, because the desire to consume caffeine is highest in those with increased screen time.

As stated above, there is a paucity of studies investigating the relationship between light exposure, screen time and eye health in children, therefore reasonable conclusions regarding the effect of excessive blue light or white light exposure on visual development in children require further study. The inventors have found that childhood may represent an important time for intervention via supplementation, before appreciable damage occurs to the developing retina.

### CONCLUSION

As discussed herein, children supplemented with lutein/zeaxanthin showed significant improvement in subjective measures of eye fatigue, eye strain, light sensitivity and objective measures of glare recovery time following 4 months of lutein and zeaxanthin supplementation following exposure to blue and even white light. These findings are both new and novel in the fact that no previous prior studies have been conducted to show that such supplementation can provide improvements in visual performance parameters amongst children. The fact that similar effects have been shown in adult subjects (i.e., individuals over the age of 18) cannot be extrapolated into children since the adult eye is fully developed whereas the eyes of children are not fully developed. Given the amount time that people spend viewing screens, especially children, in today's world, it is important to provide protection against such potential eye damaging exposures.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

What is claimed:

1. A method for reducing or minimizing the impact on visual performance resulting from the exposure to light rays comprising orally administering lutein and/or zeaxanthin to children, wherein the light rays include blue light and/or white light.

2. The method of claim 1 wherein the white light comprises wavelengths ranging from 380-750 nm.

3. The method of claim 1 wherein the blue light comprises wavelengths ranging from 380-500 nm.

4. The method of claim 1 wherein the white light or the blue light can be from a natural source or an artificial source.

5. The method of claim 1 wherein the children range from toddlers to teenagers.

6. The method of claim 1 wherein the children range in age from 2 to 18 years of age.

7. The method of claim 5 wherein the children range in age from 7 to 12 years of age.

8. The method of claim 1 wherein the impact on visual performance is due at least in part to eye strain and/or fatigue following exposure to white light or blue light.

9. The method of claim 1 wherein the impact on visual performance is due at least in part to sensitivity to light following exposure to white light or blue light.

10. The method of claim 1 wherein reducing or minimizing the impact on visual performance refers to improvement of glare recovery.

11. A method for reducing or minimizing eye strain and/or eye fatigue following exposure to light rays including blue light and/or white light, comprising orally administering lutein and/or zeaxanthin to children.

12. The method of claim 11 wherein the white light comprises wavelengths ranging from 380-750 nm.

13. The method of claim 11 wherein the blue light comprises wavelengths ranging from 380-500 nm.

14. The method of claim 11 wherein the light rays can be from a natural source or an artificial source.

15. The method of claim 11 wherein the children range from toddlers to teenagers.

16. The method of claim 11 wherein the children range in age from 2 to 18 years of age.

17. The method of claim 16 wherein the children range in age from 7 to 12 years of age.

18. A method for protecting the eyes of children from damage caused by exposure to light rays comprising orally administering lutein and/or zeaxanthin to children.

19. The method of claim 18 wherein the light rays, including blue and/or white light, can be from a natural source or an artificial source.

20. The method of claim 18 wherein children range from toddlers to teenagers.
